(19) United States
(12) Patent Application Publication
(10) Pub. No.: US 2023/0191293 A1
(43) Pub. Date: Jun. 22, 2023

(54) MULTILAYER FIBER COMPOSITE FILTER MEDIA

(71) Applicant: Donaldson Company, Inc., Minneapolis, MN (US)

(72) Inventors: Klenton T. Willis, Woodbury, MN (US); Jared R. Moody, Minneapolis, MN (US); Aflal Rahmathullah, Savage, MN (US); Anil C. Suthar, Eden Prairie, MN (US); Elizabeth Jackson, Minneapolis, MN (US)

(21) Appl. No.: 18/086,437

(22) Filed: Dec. 21, 2022

Related U.S. Application Data
(60) Provisional application No. 63/292,724, filed on Dec. 22, 2021.

(51) Int. Cl. B01D 39/16; B01D 35/31

(52) U.S. Cl. CPC B01D 39/1623; B01D 35/31; B01D 2239/0618; B01D 2239/065; B01D 2239/1216; B01D 2239/1233; B01D 2221/10

(57) ABSTRACT

A composite media includes a stack with 20 or more layers, each layer including a substrate with a first major surface and an opposing second major surface; and a fiber layer deposited onto the first major surface of the substrate and including polymeric fibers with a diameter of 100 nm to 1.5 μm. Each fiber layer independently has a thickness of 5 μm to 100 μm, and each layer independently has a pore size of 0.1 μm to 10 μm. A filter may include a housing and the composite media disposed within the housing. The filter may be a syringe filter.

### CROSS REFERENCE TO RELATED APPLICATIONS

This application claims the benefit of U.S. Provisional Application No. 63/292,724, filed 22 Dec. 2021, the disclosure of which is incorporated by reference herein in its entirety.

### FIELD

The present disclosure relates to filter media containing fine fibers. The present disclosure further relates to filter media including a plurality of layers containing fine fibers.

### SUMMARY

A composite media includes a stack with 20 or more layers, each layer including a substrate with a first major surface and an opposing second major surface; and a fiber layer deposited onto the first major surface of the substrate and including polymeric fibers with a diameter of 100 nm to 1.5 μm. Each fiber layer independently has a thickness of 5 μm to 100 μm. Each layer independently has a P95 pore size of 0.1 μm to 10 μm. The substrate may be a non-woven substrate. The substrate may be a membrane.

The stack may include a layer with two or more fiber layers. Each layer of the stack may optionally include two or more fiber layers. The stack may include a layer where a second fiber layer is deposited on the second major surface of the substrate.

The P95 pore size of the fiber layer may be from 0.5 μm to 5.0 μm, from 0.7 μm to 2.0 μm, or from 0.8 μm to 1.5 μm. The pore sizes of the fiber layers may form a gradient of pore sizes throughout the stack. The composite media may have an inlet side and an outlet side, and the gradient of pore sizes may extend from a largest P95 pore size of 50 μm adjacent the inlet side to a smallest P95 pore size of 0.1 μm adjacent the outlet side.

The stack may have a pore size of 0.01 μm to 2.5 μm. The stack may include 100 to 3000 layers, 300 to 2000 layers, or 500 to 1000 layers.

The stack may include a first fiber layer with a first composition and a second fiber layer with a second composition different from the first composition. The fiber layers of the stack may form a gradient of chemical composition throughout the stack. Alternatively, each of the layers in the stack may have the same composition and construction.

The stack may include a first plurality of layers that are under a first level of compression and a second plurality of layers that are under a second level of compression different from the first level of compression. The pluralities of layers may be under a gradient of compression levels throughout the stack.

The composite media may have an initial water flux of 150 mL/cm²/hour/kPa to 300 mL/cm²/hour/kPa.

The fiber layers may have a surface area to volume ratio of 1 μm⁻¹ or greater, and optionally 20 μm⁻¹ or less.

A filter may include a housing and the composite media disposed within the housing. The filter may be a syringe filter.

The filter housing may be constructed to apply a first level of compression on a first plurality of layers and a second level of compression on a second plurality of layers, wherein the second level of compression is different from the first level of compression. The pluralities of layers may be under a gradient of compression levels throughout the stack.

### BRIEF DESCRIPTION OF FIGURES

FIG. 1A is a schematic diagram of a single layer according to an embodiment.

FIG. 1B is a schematic diagram of a single layer according to another embodiment.

FIG. 2 is a schematic diagram of a composite media including a stack of 20 or more layers of FIG. 1A according to an embodiment.

FIG. 3 is a schematic cross-sectional view of a filter including the composite media of FIG. 2 according to an embodiment.

FIG. 4 is a schematic cross-sectional view of a filter including stacks of layers under various levels of compression according to an embodiment.

FIG. 5 is a schematic top view of a single layer according to an embodiment.

FIG. 6 is a schematic top view of a single layer with two types of fine fiber layers according to an embodiment.

FIG. 7 is a schematic depiction of two of the layers of FIG. 6 being arranged in a stack according to an embodiment.

FIGS. 8A-8D are schematic top views of stacks of two of the layers of FIG. 6 in various arrangements.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

Herein, “resin” or “resinous” refers to monomers, oligomers, and/or polymers, particularly of a nature that can migrate to the surface of a fine fiber during fiber formation.

The term “aromatic ring” is used in this disclosure to refer to a conjugated ring system of an organic compound. Aromatic rings may include carbon atoms only, or may include one or more heteroatoms, such as oxygen, nitrogen, or sulfur.

The term “alkylated” is used in this disclosure to describe compounds that are reacted to replace a hydrogen atom or a negative charge of the compound with an alkyl group, such that the alkyl group is covalently bonded to the compound.

The term “alkyl” is used in this disclosure to describe a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, etc.

The term “fine fiber” is used here to refer to a fiber that has an average fiber diameter of 10 μm or less. Typically, this means that a sample of a plurality of fibers of the present disclosure has an average fiber diameter of less than 10 μm.

The term “fiber diameter” is used here to refer to the average diameter of fibers, indicating that in a sample of a plurality of fibers, the fiber diameter is the indicated average fiber diameter. Fiber diameter may be measured using a top-down SEM image. The sample may be sputter-coated with a sputter-coater, such as a gold and palladium mixture including, for example, a 60:40 Au:Pd mixture. A more accurate fiber diameter measurement may be obtained by measuring the diameter of the fiber in at least 30 locations in the sample. Software such as a Trainable Weka Segmentation (an ImageJ plug-in) may be useful for analyzing fiber diameters.

The term “pore size” is used here to refer to a mean flow pore size.

As used here, unless indicated otherwise, pore size (for example P5, P50, and P95) is determined using capillary flow porometry. Capillary flow porometry may be performed using a continuous pressure scan mode. It may be useful to use silicone oil, having a surface tension of 20.1 dynes/cm and a wetting contact angle of 0, as a wetting liquid. The sample may initially be tested dry, varying low pressure to high pressure, and then tested wet, again varying low pressure to high pressure. The test is typically performed at ambient temperature conditions (for example, 20° C. to 25° C.). A number of data points, such as 256 data points, may be collected across the range of the scan of the pressures for both the dry curve and the wet curve. Typically, no tortuosity factor and/or a shape factor will be used (that is, for comparison to other test methods that use an adjustment factor, a factor equal to 1 may be used). As used herein, a value P(x) is the calculated pore size when the wet curve is equal to (100-x) % of the dry curve, as determined using the methodology described herein. Although a calculated value, this can be understood as representing the point at which x % of the overall flow through the layer passes through pores of that size or below. For example, P50 (the mean flow pore size) represents the point at which the wet curve is equal to half the dry curve, and may be viewed as the pore size such that 50% of the total flow through the layer is through pores of that size or below. P95 represents the point at which 95% of the total flow through the layer passes through pores of that size or below.

An average pore size (for example, average maximum pore size) may be calculated from the mean of at least three measurements (taken from at least three different sample locations.

Individual measurements of maximum pore size (which may also be referred to as P100) may be detected at the bubble point, where the bubble point is found after fluid begins passing through the sample, and three consecutive measurements increase by at least 1%, where 256 data points are collected across the scan at a rate of approximately 17 data points per minute.

The acronym SAVR is used here to refer to the surface area to volume ratio of a material. SAVR may be either estimated or calculated. SAVR may be estimated as SAVR=(4*c)/df, where c is the solidity of the material (e.g., fiber layer) and df is the mean fiber diameter. SAVR may be calculated as SAVR=(Length of fiber*circumference of fiber)/Volume of the layer. SAVR is typically calculated for a specific area of the material, such as a 1 inch (25 mm) diameter die cut piece of material.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood to have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to filter media containing fine fibers. The present disclosure further relates to filter media including a plurality of layers containing fine fibers.

According to an embodiment, a composite media includes a stack including layers, where each layer includes a substrate with a first major surface and an opposing second major surface; and a fiber layer deposited onto the first major surface of the substrate. The fiber layer includes polymeric fibers. The fibers may be fine fibers or nanofibers. The fibers may have a diameter of 100 nm to 1.5 μm. The fiber layers may have a thickness 5 μm to 100 μm. The layers may have a P95 pore size of 0.1 μm to 10 μm.

According to an embodiment, the composite media (e.g., the fiber layers of the composite media) has a high surface area to volume ratio (“SAVR”). The composite media may be used in any filter that may benefit from the layered fine fiber structure and high SAVR. In some cases, the composite media is used in a syringe filter.

The number of layers in the stack may be varied to add depth and consequently different properties to the composite media. The composite media may exhibit differences between the layers of the stack. The composite media may include multiple stacks with different properties. The composite media may provide one or more various gradients, such as a pore size gradient, or a gradient of chemical composition and functionality.

The number of layers in the stack may be 2 or more, 5 or more, 10 or more, 20 or more, 50 or more, 100 or more, 200 or more, 300 or more, or 500 or more. The stack may include 3000 or fewer, 2500 or fewer, 2000 or fewer, 1500 or fewer, or 1000 or fewer layers. The number of layers in the stack may be 100 to 3000 layers, 300 to 2000 layers, or 500 to 1000 layers.

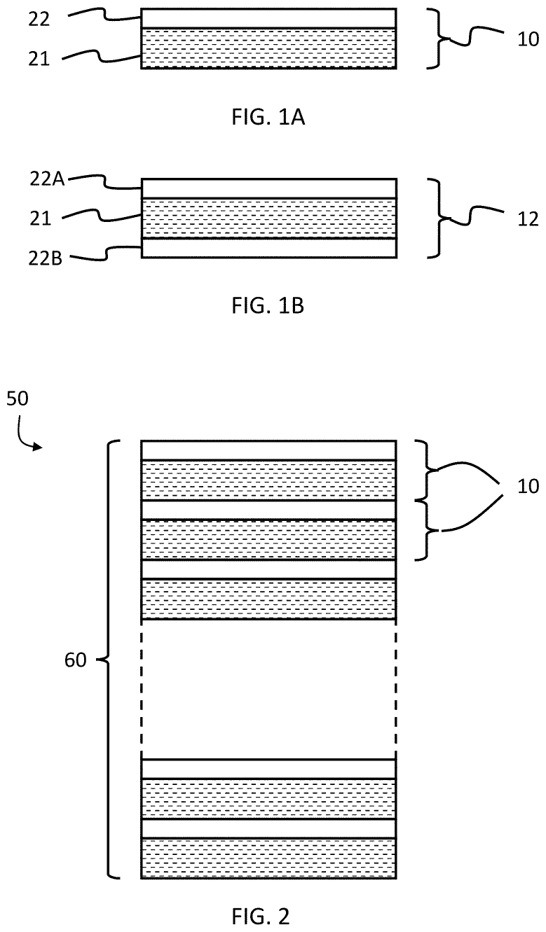

Each layer of the stack has at least one substrate and at least one fiber layer. Typically, each layer only includes one substrate. The layers may include a single substrate and a single fiber layer deposited onto the substrate. Referring now to FIG. 1A, a schematic diagram of a single layer 10 is shown, including a substrate 21 and a fiber layer 22 deposited onto the substrate 21. A schematic diagram of a composite media 50 including a stack 60 of 20 or more of the layers 10 is shown in FIG. 2.

In some embodiments, one or more of the layers may include a single substrate and two or more fiber layers deposited onto the substrate. For example, one or more of the layers may include a single substrate and one fiber layer deposited on the first major side of the substrate and another fiber layer deposited on the second major side of the substrate. Referring now to FIG. 1B, a schematic diagram of a single layer 12 is shown, including a substrate 21 and a first fiber layer 22A deposited onto the first major side of the substrate 21 and a second fiber layer 22B deposited onto the second major side of the substrate 21.

The layers may also include a first fiber layer deposited onto the substrate and one or more additional fiber layers deposited onto the first fiber layer. Such fiber layers may be distinguished from one another, for example, by their structure or chemical composition.

According to an embodiment, the fiber layer includes polymeric fibers. That is, the fibers include or consist of polymeric materials. In some embodiments, the fiber layer is free of glass fibers. In some embodiments, the polymeric materials are selected such that they are suitable for deposition by electrospinning. The composition of the polymeric fibers may also be selected to impart the fiber layer desirable properties, such as chemical affinity, based on the intended use of the composite media.

The fibers may have a diameter of 100 nm or greater, 200 nm or greater, 300 nm or greater, 400 nm or greater, or 500 nm or greater. The fibers may have a diameter of 3 μm or less, 2 μm or less, 1.5 μm or less, 1.2 μm or less, or 1.0 μm or less. In some embodiments, the fibers have a diameter ranging from 100 nm to 1.5 μm, from 300 nm to 1.2 μm, or from 500 nm to 1.0 μm. In some cases, the fibers may be characterized as fine fibers. The fiber diameters in each of the layers are not necessarily the same throughout the stack. The fiber diameter of the layers may be independently selected. The layers within the stack may form a fiber diameter gradient extending throughout the stack.

Each fiber layer of the stack may independently have a thickness of 5 μm or greater, 10 μm or greater, 25 μm or greater, or 50 μm or greater. Each fiber layer may independently have a thickness of 100 μm or less, 80 μm or less, or 60 μm or less. The thickness of the fiber layer may range from 5 μm to 100 μm, 10 μm to 80 μm, or from 20 μm to 60 μm. The thickness of each of the fiber layers is not necessarily the same throughout the stack. Different fiber layers within the stack may have different thicknesses. The fiber layers within the stack may form a gradient of fiber layer thicknesses extending throughout the stack.

The substrate may have a thickness of 50 μm or greater, 100 μm or greater, or 500 μm or greater. The substrate may have a thickness of 1000 μm or less, 750 μm or less, or 500 μm or less. The thickness of the substrate may range from 50 μm to 1000 μm, or from 100 μm to 500 μm. The total thickness of the layer (combined substrate and fiber layer) may be from 55 μm to 1100 μm or from 150 μm to 600 μm. Since the filter media may include a stack of 100 to 3000 layers, the total thickness of the filter media may be 5500 μm (5.5 mm) or greater.

Each layer may independently have a P95 pore size of 0.05 μm or greater, 0.1 μm or greater, 0.3 μm or greater, 0.5 μm or greater, 0.7 μm or greater, or 1.0 μm or greater. Each layer may independently have a P95 pore size of 20 μm or less, 10 μm or less, 7.5 μm or less, 5.0 μm or less, 3.0 μm or less, 2.0 μm or less, or 1.5 μm or less. The P95 pore size of the layer may be in a range from 0.1 μm to 10 μm, 0.5 μm to 5.0 μm, from 0.7 μm to 2.0 μm, from 0.8 μm to 1.5 μm, or from 1.0 μm to 1.5 μm. The pore sizes of each of the layers is not necessarily the same throughout the stack. Different layers within the stack may have different pore sizes. The layers within the stack may form a gradient of layer pore sizes extending throughout the stack. According to an embodiment, the composite media has a high SAVR. The fiber layer may have a SAVR of 1 μm⁻¹ or greater, 2 μm⁻¹ or greater, or 5 μm⁻¹ or greater. The fiber layer may have SAVR of 20 μm⁻¹ or less or 15 μm⁻¹ or less. The SAVR of the fiber layer may range from 1 μm⁻¹ to 20 μm⁻¹ or from 2 μm″¹ to 15 μm⁻¹.

The substrate may be made of any suitable material. In some embodiments, the substrate is a non-woven substrate. In some embodiments, the substrate comprises a membrane.

The stack as a whole may have a P95 pore size of 0.01 μm or greater, 0.05 μm or greater, 0.1 μm or greater, 0.3 μm or greater, 0.5 μm or greater, 0.75 μm or greater, or 1.0 μm or greater. The stack may have a P95 pore size of 2.5 μm or less, 2.2 μm or less, 2.0 μm or less, 1.75 μm or less, or 1.5 μm or less, of 1.0 μm or less. The P95 pore size of the stack may be from 0.01 μm to 2.5 μm, from 0.1 μm to 2.0 μm, or from 0.5 μm to 1.5 μm.

According to an embodiment, the composite media has an initial water flux of 100 mL/cm²/hour/kPa or greater, 120 mL/cm²/hour/kPa or greater, 150 mL/cm²/hour/kPa or greater, 175 mL/cm²/hour/kPa or greater, or 200 mL/cm²/hour/kPa or greater. The initial water flux may be 350 mL/cm²/hour/kPa or less, 300 mL/cm²/hour/kPa or less, or 250 mL/cm²/hour/kPa or less. Water flux is measured by applying water at 25° C. and 1 bar to the composite media and measuring the flow rate with a flow meter. Measured values include the pressure, water temperature, and volumetric flow rate of the water.

Any suitable polymer or combination of polymers may be used to prepare the polymeric fibers of the fiber layer. The composition of the fiber layers may be independently selected. The material of a single fiber may be homogenous (e.g., a polymer, copolymer, or blend of polymers) throughout the fiber, or may include a core-sheath structure, where the core has a different composition than the sheath. Suitable polymeric materials are described, for example, in WO2013/043987 filed on 21 Sep. 2012, and WO2014/164130 filed on 5 Mar. 2014, both of which are incorporated by reference herein in their entireties.

In some embodiments, the polymeric fibers are formed by mixing or blending a polymer material (e.g., a fiber-forming polymer) with a resinous aldehyde composition. In certain embodiments, the aldehyde composition is a melamine-aldehyde composition. When formed into a fiber, the mixture or blend of fiber-forming polymer material and resinous aldehyde composition, at appropriate ratios, may form at least two (e.g., concentric or coaxial) phases. The first phase is an internal core that includes the fiber-forming polymer material. The core is surrounded by a second phase (a coating) that includes the resinous aldehyde composition. A proportion of the resinous aldehyde may crosslink adjacent polymer chains residing in the core. A transition layer or transition phase including a mixture or blend of the polymer material and a resinous aldehyde may be formed between the core and the coating. The weight ratio of the resinous aldehyde composition to the polymer may be 20 parts or more by weight resinous aldehyde composition per 100 parts by weight of the polymer. The fiber-forming polymer material may also include reactive groups, such as functional groups (e.g., active hydrogen groups) capable of being crosslinked by the alkoxy groups of the resinous aldehyde composition.

The term “fiber-forming polymer” (e.g., homopolymer or copolymer) is used to refer to a polymer that is capable of forming a fine fiber in the absence of reactive additives.

The fiber-forming polymer material and the resinous aldehyde composition may be combined in solution or melt form. In certain embodiments, the polymeric fibers are electrospun from a solution or dispersion. Thus, the polymer material and resinous aldehyde (e.g., melamine-aldehyde) compositions are dispersible or soluble in at least one common solvent or solvent blend suitable for electrospinning.

Suitable resinous aldehyde compositions include two or more alkoxy groups per molecule that are capable of crosslinking a polymer used in making the fine fibers as described herein. Exemplary resinous aldehyde compositions useful as crosslinking agents include a condensation product of urea and an aldehyde, a condensation product of phenol and an aldehyde, or a condensation product of melamine and an aldehyde. One useful class of crosslinking resins includes resins based on nitrogen compounds such as melamine, urea, benzoguanamine, glycoluril, and other similar resins manufactured by reacting an aldehyde with a nitrogen compound.

Useful resinous aldehyde compositions (e.g., melamine-aldehyde compositions) include compounds and mixtures thereof including: highly methylated melamine; partially methylated melamine; methylated high imino melamine; highly alkylated mixed ether melamine; highly alkylated carboxylated, high imino mixed ether melamine; highly n-butylated melamine; n-butylated high imino and partially n-butylated melamine; partially iso-butylated melamine; partially n-butylated urea; partially iso-butylated urea; glycoluril; highly alkylated mixed ether melamine-formaldehyde; highly alkylated mixed ether carboxylated melamine resin; hexabutoxymethyl melamine; butoxy methyl melamine; highly alkylated mixed ether melamine; methoxymethyl methylol melamine, highly methylated melamine resins; melamine-formaldehyde resin co-etherified with methanol and n-butoxy ethanol/n-butanol blend; melamine-formaldehyde resin co-etherified with methanol and n-butanol in n-butanol; butylated melamine-formaldehyde resin dissolved in a blend of n-butanol and butyl glycol; partially n-butylated melamine; high solids, highly methylated melamine resins; various resinous aldehyde compositions sold under the trade names CYMEL available from Allnex GmbH in Frankfurt am Main, Germany; various resinous aldehyde compositions sold under the trade name LUWIPAL and available from the BASF AG of Ludwigshafen, Germany; resins sold under the trade names RESIMENE, MAPRENAL, and MADURIT available from Prefere Resins Holding GmbH in Erkner, Germany. Various combinations of resinous aldehyde compositions can be used if desired. The term “highly” in the context of degree of substitution (e.g., highly alkylated, highly methylated, etc.) is understood to mean 50% or greater. That is, 50% or more of the available substitution sites are substituted.

Preferred fiber-forming polymer materials include one or more active hydrogen groups capable of reacting with and crosslinking to the resinous aldehyde compositions. Active hydrogen groups include, but are not limited to, thiol (—SH), hydroxyl (—OH), carboxylate (—CO₂H), amido (—C(O)—NH— or —C(O)—NH₂), amino (—NH₂), or imino (—NH—), and anhydride (—COO)₂R groups (upon hydrolysis). Polymer materials suitable for use in the polymeric compositions of the disclosure include both addition polymer and condensation polymer materials with active hydrogens. Suitable examples include poly(meth)acrylic acids, polyamides, cellulose ethers and esters, poly(maleic anhydride), polyamines such as chitosan and mixtures, blends, alloys, and block, graft, or random copolymers thereof. Preferred materials that fall within these generic classes include poly(vinyl alcohol) in various degrees of hydrolysis (e.g., 87% to 99.5%) in crosslinked and non-crosslinked forms. Other preferred examples of useful polymer materials include cellulose derivatives selected from the group consisting of ethyl cellulose, hydroxyl ethyl cellulose, cellulose acetate, cellulose acetate butyrate, cellulose acetate propionate, cellulose acetate phthalate, and mixtures thereof; poly(meth)acrylic acid homopolymers and copolymers, including for example, styrene-(meth)acrylic acid copolymers and ethylene-(meth)acrylic acid copolymers; polyvinyl alcohol homopolymers or copolymers, including for example, a polyvinyl butyral and an ethylene co-vinyl alcohol copolymer; poly(maleic anhydride) homopolymers or copolymers, including for example, a styrene-maleic anhydride copolymer; and polyurethanes. Herein, a poly(meth)acrylic acid refers to poly(acrylic acid) and poly(methacrylic acid) polymers.

Many types of polyamides are also useful as the polymer materials in the fibers of the disclosure. One useful class of polyamide condensation polymers are nylon materials. The term “nylon” is a generic name for all long chain synthetic polyamides. Typically, nylon nomenclature includes a series of numbers such as in nylon-6,6 which indicates that the starting materials are a C6 diamine and a C6 diacid (the first digit indicating a C6 diamine and the second digit indicating a C6 dicarboxylic acid compound). Another nylon can be made by the polycondensation of ε-caprolactam in the presence of a small amount of water. This reaction forms a nylon-6 (made from a cyclic lactam, also known as ε-aminocaproic acid) that is a linear polyamide. Further, nylon copolymers are also contemplated. Exemplary nylon materials include nylon-6, nylon-6,6, nylon-6,10, mixtures or copolymers thereof.

Copolymers can be made by combining various diamine compounds, various diacid compounds and various cyclic lactam structures in a reaction mixture and then forming the nylon with randomly positioned monomeric materials in a polyamide structure. For example, a nylon-6,6-6,10 material is a nylon manufactured from hexamethylene diamine and a C6 and a C10 blend of diacids. A nylon-6-6,6-6,10 is a nylon manufactured by copolymerization of ε-aminocaproic acid, hexamethylene diamine and a blend of a C6 and a C10 diacid material. Herein, the term “copolymer” includes polymers made from two or more different monomers and include terpolymers, etc.

Block copolymers are also useful as the polymer materials in the fibers of the disclosure. With such copolymers, where fibers will be electrospun, the choice of solvent or solvent blend is important. The selected solvent or solvent blend is selected such that both blocks are soluble in the solvent. Examples of useful block copolymers include PEBAX ε-caprolactam-b-ethylene oxide, available from Arkema Inc. of Philadelphia, Pa.; and polyurethanes of ethylene oxide and isocyanates.

Addition polymers like polyvinyl alcohol, and amorphous addition polymers such as poly(acrylonitrile) copolymers with acrylic acid are also useful. They can be solution spun with relative ease because they are soluble or dispersible in a variety of solvents and solvent blends at low pressures and temperatures. A poly(vinyl alcohol) having a hydrolysis degree of, for example, from 87 to 99.9+% can be used as the polymer material in the fibers of the disclosure.

Preferred polymers include a polyamides (particularly nylon), polyester amides, a polyvinyl alcohol, an ethylene-co-vinyl alcohol polymer, a polyvinyl butyral, and poly(maleic anhydride). Preferred active hydrogen groups include hydroxy-1, amino, and amido groups. Various combinations of polymer materials can be used if desired.

In some embodiments, the polymeric fiber may be formed from the fiber-forming polymer material and at least two reactive additives that are capable of reacting with each other, for example, in a fiber-forming process or in a post-treatment process. The at least two reactive additives are optionally also reactive with the fiber-forming polymer. Typically, the amount of reactive additive(s) relative to the fiber-forming polymer(s) is 0.5 parts or more, 1 part or more, 5 parts or more, 10 parts or more, or 20 parts or more, by weight, or reactive additive per 100 parts by weight of the fiber-forming polymer. Typically, the amount of reactive additive(s) relative to the fiber-forming polymer(s) is 200 parts or less, or 50 parts or less, by weight, or reactive additive per 100 parts by weight of the fiber-forming polymer.

The term “reactive additive” refers to monomers, oligomers, and/or polymers, that include functional groups capable of reacting with functional groups of other reactive additives, and optionally with the fiber-forming polymer.

The reactive additive may have a weight average molecular weight of less than 3000 Daltons. Preferred reactive additives are substantially nonvolatile at room temperature and pressure. Reactive additives are selected such that they are preferably soluble in a solvent chosen for the polymer material for processing, such as in electrospinning. The reactive additive may be a surface-migrating agent, capable of migrating to the surface of a fine fiber, typically during fiber formation. The following are examples of various suitable reactive additives categorized by functional groups: alkoxy-functional; hydroxyl-functional; acid-functional; glycidyl ether-functional; isocyanate-functional; amine-functional; and dichloro-functional. Various reactive combinations (e.g., combinations of materials that are reactive with each other) can be used in making fine fibers of the present disclosure.

Suitable alkoxy-functional reactive additives include the exemplary resinous aldehyde compositions (e.g., melamine-aldehyde compositions) discussed above.

Hydroxyl-functional reactive additives may include Bisphenol A, Bisphenol AF, 4,4(1,4-phenylenediisopropylidene) bisphenol; (PDPBPA), 4,4′(1-phenylethlyidene) bisphenol; (PEDBPA), hydroxyl group containing antioxidants that are commonly used in polymer processing such as hindered aromatic phenols, those available under the tradenames HOSTANOX O3 (from Clariant), IRGANOX, etc., fluorinated diols such as POLYFOX reactive polymer intermediates from Omnova Chemicals, hydroxyl containing compounds available under the tradenames FOMBLIN PFPE FUNCTIONAL and FLUOROLINK from Solvay, aliphatic polycarbonate diols (e.g., that available under the tradename M112 from Perstop), phenoxy resins, phenolic resins, novolac resins, resorcinol, and polyols. If a polyol is used, such polyol preferably includes at least 2 hydroxyl groups, and often up to 20 hydroxyl groups, per every molecule of polyol. Hydroxyl-functional reactive additives may include hydroxyl-functional unsaturated monomers, such as (meth)acrylated pentaerythritol derivatives, (meth)acrylated glycerol, (meth)acrylated trimethylol propane, (meth)acrylated DGEBA, unsaturated polyesters, hydroxyethyl methacrylate, hydroxy alkyl (meth)acrylate, allyl alcohol propoxylate, allyl ethers and esters of polyhydric alcohols such as allyl ethers of trimethylol propane (e.g., 1,1,1-trimethylolpropane) or pentaerythritol or glycerol, erythritol, threitol, pentaerythritol, sorbitol, etc. Hydroxyl-functional reactive additives can include cycloaliphatic polyols such as cyclohexane dimethanol (e.g., the diol UNOXOL available from Dow) or ethoxylates thereof, ethoxylated or propoxylated polyhydric alcohols (e.g., those available under the tradename BOLTRON polyols and ethoxylated pentaerythritol from Perstrop). They can include heterocyclic-based polyols. They can include copolymers of unsaturated aromatic monomers, such as styrene, and hydroxyl-containing unsaturated monomers, such as styrene-allyl alcohol copolymers available under the tradename SAA from Lyondell Corp.

Hydroxyl-functional reactive additives can include polymers containing hydroxyl groups such as polyvinyl alcohol, polymers and copolymers containing hydroxyl groups such as ethylene vinyl alcohol, polyvinyl butyral, and cellulose acetate, cellulose acetate butyrate, cellulose acetate propionate, etc.

Acid-functional reactive additives may include diacids, triacids, etc. Typically, they are carboxylic acids, such as glutaric acid, succinic acid, adipic acid, malonic acid, pimelic acid, suberic acid, azelaic acid, sebacic acid, undecanedioic acid, dodecanedioic acid, phthalic acid, terephthalic acid, isophthalic acid, maleic acid, fumaric acid, glutaconic acid, traumatic acid, muconic acid, citric acid, ascorbic acid, dimethylolpropionic acid, fluorinated acids such as acid-containing compounds such as those available under the tradenames FOMBLIN PFPE FUNCTIONAL and FLUOROLINK from Solvay, polycarboxylic acids such as polyacrylic acids, styrene acrylic acid, polymethacrylic acid, styrene methacrylic acid, etc. Acid-functional reactive additives may also include acyl halides, such as adipoyl chloride. Acid-functional reactive additives may include weakly acidic oligomers or polymers. Examples include (meth)acrylic acid (i.e., acrylic acid or methacrylic acid) copolymers with other unsaturated monomers such as styrene, maleic acid or anhydride copolymer with other unsaturated monomers such as styrene (e.g., styrene-maleic anhydride copolymer), graft polymers wherein the graft group is a carboxylic acid or anhydride thereof, polymers that include phosphoric acid and esters thereof (e.g., the additive available under the tradename ADDITOL XL-180 from Allnex GmbH), and unsaturated polycarboxylic resins characterized by dual functionality (e.g., that available under the tradename SARBOX SB500E 50 from Sartomer).

Glycidyl ether-functional reactive additives may include diglycidyl ether-containing additives such as trimethylolpropane triglycidyl ether, as well as resins sold as epoxy resins or low molecular weight reactive epoxy resins or diluents. Representative examples include epoxy resins, modified epoxy resins, brominated epoxy resins, epoxy reactive diluents sold under the tradename D.E.R by Dow Chemicals. These include Bisphenol A diglycidyl ether, Bisphenol A/F diglycidyl ether, Bisphenol F diglycidyl ether, and modified Bisphenol A diglycidyl ether, Bisphenol A/F diglycidyl ether, Bisphenol F diglycidyl ether, etc. Other examples include epoxy novolac resins sold under the tradename D.E.N. by Dow Chemicals and epoxy resins such as that sold by Momentive. These include epoxy resins, and epoxy multi-functional resins, epoxy novolac resins, and epoxy polyacrylate resins sold under the tradename EPON, epoxy functional modifiers sold under the tradename HELOXY, and cycloaliphatic epoxy resin sold under the tradename EPONEX.

Isocyanate-functional reactive additives may include aliphatic and aromatic polyisocyanates (e. g., triphenylmethane triisocyanate), preferably aromatic and aliphatic blocked polyisocyanates, such as those sold by Bayer under tradenames DESMODUR BL, BAYHYDUR BL, EVONIK, and VESTANAT.

Amine-functional reactive additives may include polyethyleneimine, chitosan, lysine, polylysine, amino acids, and amines including phenylene diamine (para, ortho, meta), dimethyl 4-phenylene diamine, triethylenetetramine, trimethylol propane tris(poly(propylene glycol) amine terminated) ether. Amine-functional reactive additives may also include aliphatic and aromatic polyurethanes, fluorinated polyurethanes (prepolymers, oligomeric and polymeric).

An example of a dichloro-functional reactive additive is dichlorodiphenyl sulfone.

In some embodiments, individual fiber layers include variations in the structure, chemical composition, or properties of the fibers within the individual fiber layer. The features that may be varied may include, for example, fiber diameter, pore size, solidity, and chemical composition of the fibers. Variations in any one feature (e.g., fiber diameter) may be combined with variations in another feature (e.g., chemical composition). For example, the fiber layer may include first fibers with a first composition and second fibers with a second composition that is different from the first composition. The fiber diameters of the first and second fibers may also be different.

According to an embodiment, the fibers of the fiber layer include variations in fiber diameter. That is, the fiber layer includes fibers with a first fiber diameter and fibers with a second fiber diameter. The fiber layer may further include fibers with a third, fourth, and subsequent fiber diameters. The fiber layer may include a mixture of the fibers with different fiber diameters. In some embodiments, the fiber layer includes a gradient of fiber diameters.

According to an embodiment, the fibers of the fiber layer include variations in chemical composition. That is, the fiber layer includes fibers with a first chemical composition and fibers with a second chemical composition. The fiber layer may further include fibers with a third, fourth, and subsequent chemical composition. The fiber layer may include a mixture of the fibers with different chemical compositions. In some embodiments, the fiber layer includes a gradient of chemical compositions. Materials for functionalized or chemical filtration may include fibers made of polymers with select functional groups or other materials, such as activated carbon or other adsorbents or absorbents, included with the polymeric material. The fiber layer may include a mixture of the fibers with different properties due to the different chemical compositions of the fibers.

In some embodiments, individual fiber layers include variations in the structure, chemical composition, or properties of the fibers within the individual fiber layer. The features that may be varied may include, for example, fiber diameter, pore size, solidity, and chemical composition of the fibers. Variations in any one feature (e.g., fiber diameter) may be combined with variations in another feature (e.g., chemical composition). For example, the fiber layer may include first fibers with a first composition and second fibers with a second composition that is different from the first composition. The fiber diameters of the first and second fibers may also be different.

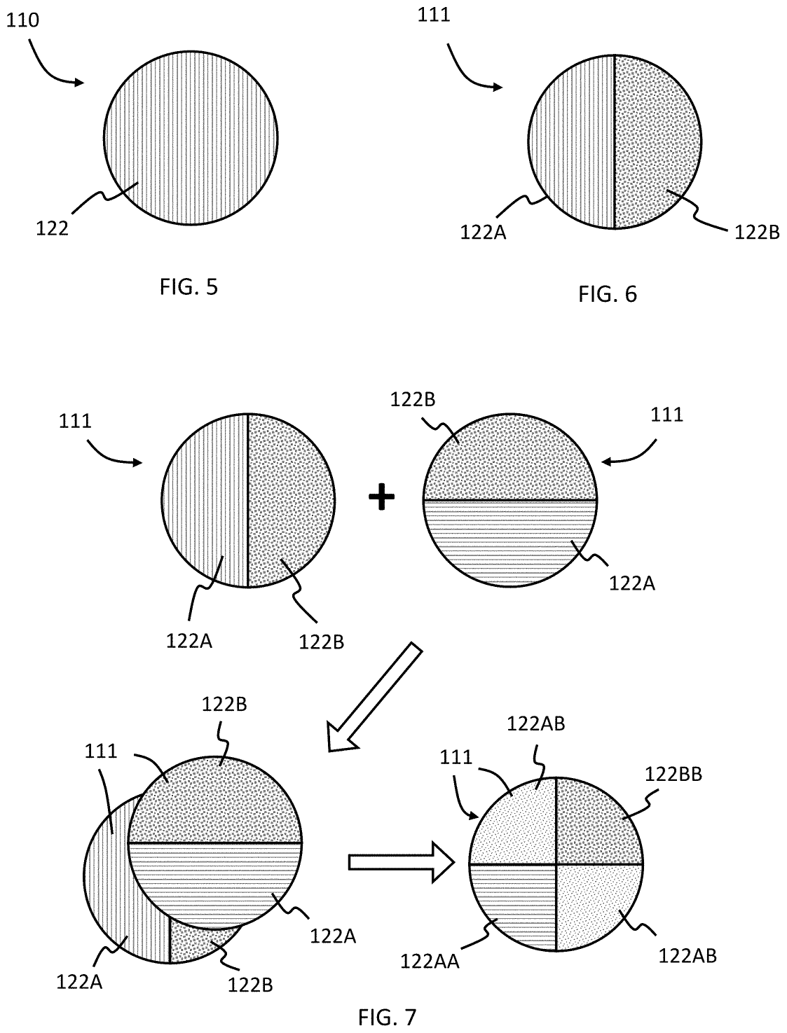

In some embodiments, the variations in one or more features within a fiber layer are arranged in a side-by-side configuration. FIG. 5 illustrates is a schematic top view of a single layer 110 with a homogeneous fiber layer 122. FIG. 6 illustrates is a schematic top view of a single layer 111 with a first type of fiber layer 122A adjacent a second type of fiber layer 122B. The first and second types of fiber layers 122A, 122B together form the fiber layer. The first and second types of fiber layers 122A, 122B may be arranged in a pattern or be randomly dispersed in the layer 111. The first and second types of fiber layers 122A, 122B may constitute equal portions of the layer 111 or one type may make up a larger portion than the other. Fine fiber layers including first and second types of fiber layers 122A, 122B may be sandwiched between two substrate layers, forming a substrate-fine fiber layer-fine fiber layer-substrate (“S-FF-FF-S”) configuration.

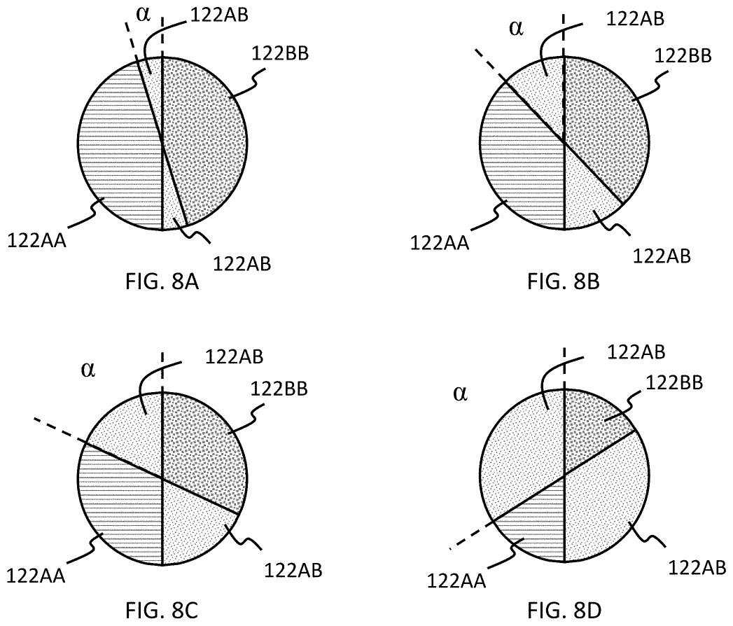

Two or more of the layers 111 may be stacked together as illustrated in FIG. 7. The layers may be oriented such that the first and second types of fiber layers 122A, 122B are not aligned. This creates areas where the first type of fiber layer 122A overlaps with another layer of the first type of fiber layer 122A, creating area 122AA, where the first type of fiber layer 122A overlaps with the second type of fiber layer 122B, creating area 122AB, and where the second type of fiber layer 122B overlaps with another layer of the second type of fiber layer 122B, creating area 122BB. FIGS. 8A-8D illustrates embodiments where the orientation of the layers is varied by angle α, thus changing the size (area) of the areas 122AA, 122AB, and 122BB.

The first and second types of fiber layers 122A, 122B may differ by their solidity, fiber size, pore size, chemical composition, or another property. In one exemplary embodiment, fine fiber layers include first and second types of fiber layers 122A, 122B with different solidities. The fine fiber layers are arranged in a S-FF-FF-S configuration. In areas where the two different solidities overlap, the solidity of the combined layer is dominated by the higher solidity portion but has a theoretical lower differential pressure than the portion where two higher solidity portion layers overlap. Multiple such layers with differing angle α orientations may be stacked to create a composite media with a gradient structure. Such arrangements may provide the ability to capture smaller particle sizes higher in the stack, which could increase the direct holding capacity (DHC) and life of the filter. Such arrangements may also allow increased control of flow within the configuration by increasing the dwell time of fluid being filtered.

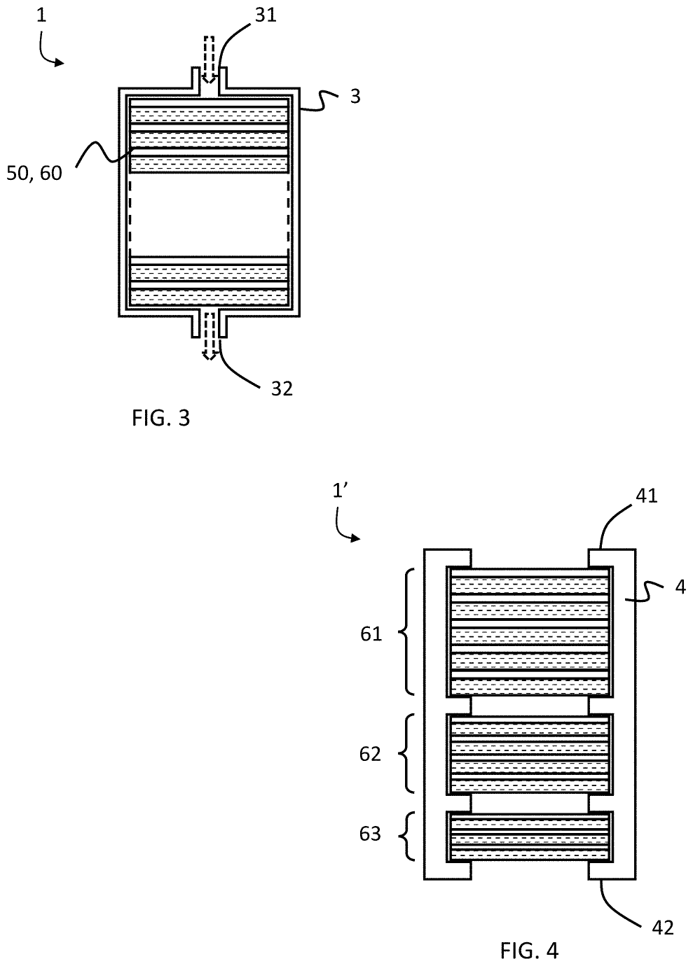

According to an embodiment, the composite media is included in a filter. Such a filter may include a housing and the composite media disposed within the housing. Referring now to FIG. 3, a schematic diagram of a filter 1 is shown, including a housing 3 and composite media 50 disposed therein. The filter housing forms an inlet 31 and an outlet 32. It is noted that the composite media 50 may be oriented within the filter housing 3 either as shown, with the fiber layer 22 oriented toward the inlet 31 and the substrate 21 oriented toward the outlet, or the opposite way with the fiber layer 22 oriented toward the outlet 32 and the substrate 21 oriented toward the inlet 31. In some cases, some layers are oriented one way (e.g., fiber layer 22 oriented toward the inlet 31) and other layers are oriented another way (e.g., substrate 21 oriented toward the inlet 31). Additional layers may optionally also be included. Any suitable filter housing may be used. The composite media may be suitable for use in filtering various liquids, such as aqueous or non-aqueous liquids. In some embodiments, the filter is a syringe filter. Typical syringe filters are small disk-shaped filters that can be attached to the end of a syringe, e.g., by a LUER-LOK connection. In some embodiments the filter is a syringe filter having an inner diameter of 25 mm or 47 mm. In some preferred embodiments, the filter is sterilizable, for example by gamma radiation.

In some embodiments, the composite media includes variations in the structure, chemical composition, or properties of the layers throughout the stack. In some embodiments, the variations form a gradient within the stack. The features that may be varied may include, for example, fiber diameter, pore size, thickness of the fiber layers, chemical composition of the fibers, and compression of the layers within the stack. Variations in any one feature may be combined with variations in one or more other features throughout the stack. For example, variations in chemical composition may be combined with variations in pore size and fiber layer thickness.

According to an embodiment, the layers of the composite media include variations in fiber diameter. That is, the composite media includes one or more layers with a first fiber diameter and one or more layers with a second fiber diameter. The composite media may further include one or more layers with a third, fourth, and subsequent fiber diameters. The layers having a given fiber diameter may be grouped together. The composite media may include a first group of layers having the first fiber diameter and a second group of layers having the second fiber diameter. In some embodiments, the composite media includes a gradient of fiber diameters.

According to an embodiment, the layers of the composite media include variations in pore sizes from one layer to another. That is, the composite media includes one or more layers with a first pore size and one or more layers with a second pore size. The composite media may further include one or more layers with a third, fourth, and subsequent pore sizes. The layers having a given pore size may be grouped together. The composite media may include a first group of layers having the first pore size and a second group of layers having the second pore size. In some embodiments, the composite media includes a gradient of pore sizes. The composite media may have an inlet side (upstream side) and an outlet side (downstream side).

For example, the composite media may be arranged in a filter with an inlet and an outlet. The composite media may include layers with a larger pore size adjacent the inlet side and layers with a smaller pore size adjacent the outlet side. In one embodiment, the composite media has one or more layers at the inlet side having a P95 pore size of 1 μm or greater, 2 μm or greater, 5 μm or greater, 10 μm or greater, or 25 μm or greater. The one or more layers at the inlet side may have a P95 pore size of 50 μm or less, 40 μm or less, 30 μm or less, 25 μm or less, 20 μm or less, 15 μm or less, 10 μm or less, or 5 μm or less. The one or more layers at the inlet side may have a P95 pore size ranging from 1 μm to 50 μm, or from 5 μm to 40 μm. In one embodiment, the composite media has one or more layers at the outlet side having a P95 pore size of 0.1 μm or greater, 0.3 μm or greater, 0.5 μm or greater, 0.75 μm or greater, or 1.0 μm or greater. The one or more layers at the outlet side may have a P95 pore size of 3.0 μm or less, 2.0 μm or less, 1.5 μm or less, 1.2 μm or less, 1.0 μm or less, 0.75 μm or less, or 0.5 μm or less. The one or more layers at the outlet side may have a P95 pore size ranging from 0.1 μm to 10 μm, 0.5 μm to 5.0 μm, from 0.75 μm to 2.0 μm, from 0.8 μm to 1.5 μm, or from 1.0 μm to 1.5 μm. In one embodiment, the composite media has a pore size gradient ranging from a largest P95 pore size of 10 μm to 50 μm at the inlet side to a smallest P95 pore size of 0.1 μm to 1 μm at the outlet side. In some embodiments, the layers with different pore sizes are arranged to form a sieving layer (e.g., a stack of multiple layers) at the inlet side of the composite media and a filtration layer (e.g., a stack of multiple layers) at the outlet side of the composite media. The filtration layer may further include fibers made materials for functionalized or chemical filtration. Materials for functionalized or chemical filtration may include polymers with select functional groups or other materials, such as activated carbon or other adsorbents or absorbents, included with the polymeric material. Alternatively, or in addition, the sieving layer, the filtration layer, or both may be made with materials that render the layer(s) hydrophobic or hydrophilic, as desired.

According to an embodiment, the layers of the composite media include variations in fiber layer thickness. That is, the composite media includes one or more layers with a first fiber layer thickness and one or more layers with a second fiber layer thickness. The composite media may further include one or more layers with a third, fourth, and subsequent fiber layer thickness. The fiber layers having a given fiber layer thickness may be grouped together. The composite media may include a first group of layers having the first fiber layer thickness and a second group of layers having the second fiber layer thickness. In some embodiments, the composite media includes a gradient of fiber layer thicknesses. In general, fiber layers with larger pore sizes may be constructed to be thicker in order to accommodate a larger volume of trapped contaminants. On the other hand, fiber layers with smaller pore sizes may be constructed to be thinner to limit the pressure drop across these layers.

According to an embodiment, the layers of the composite media include variations in chemical composition of the fibers from one layer to another. That is, the composite media includes one or more layers with a first chemical composition and one or more layers with a second chemical composition. The composite media may further include one or more layers with a third, fourth, and subsequent chemical composition. The layers having a given chemical composition may be grouped together. The composite media may include a first group of layers having the first chemical composition and a second group of layers having the second chemical composition. In some embodiments, the composite media includes a gradient of chemical compositions. The composite media may have an inlet side and an outlet side. For example, the composite media may be arranged in a filter with an inlet and an outlet. The composite media may include layers with a given chemical composition adjacent the inlet side and layers with another chemical composition adjacent the outlet side. In some embodiments, the layers adjacent the outlet side are made with or coated with a composition suitable for functionalized or chemical filtrations. Materials for functionalized or chemical filtration may include polymers with select functional groups or other materials, such as activated carbon or other adsorbents or absorbents, included with the polymeric material. In some embodiments, alternatively or in addition, some of the layers may be made with materials that render the layer(s) hydrophobic or hydrophilic, as desired. The composite media may include a gradient of hydrophobicity or hydrophilicity throughout the stack.

In some embodiments, the stack includes one or more layers that are suitable for acting as a loading layer. The loading layer may be disposed at the inlet (upstream) side of the composite media. The loading layer may include one or more fine fiber layers, each of which may be supported by a substrate. In general, a loading layer exhibits properties that are suitable for loading contaminants of interest onto the layer. For example, the loading layer may have a larger pore size than the rest of the stack. Such loading layers may be suitable for sieving. The loading layer may have a chemical composition that has affinity for the contaminant of interest. The layer or layers used as a loading layer may have a P95 pore size that is 2 μm or greater, 3 μm or greater, 4 μm or greater, or 5 μm or greater. The P95 pore size may be 20 μm or less, 15 μm or less, 12 μm or less, 10 μm or less, or 8 μm or less. The P95 pore size may be in a range from 2 μm to 20 μm, 3 μm to 15 μm, 4 μm to 10 or 5 μm to 8 μm. The pore size may be selected based on the contaminant of interest. The layer or layers used as a loading layer may have a fiber diameter of 500 nm or greater, 750 μm or greater, or 1 μm or greater. The fibers may have a diameter of 3 μm or less, 2 μm or less, 1.5 μm or less, or 1.2 μm or less. In some embodiments, the fibers have a diameter ranging from 500 nm to 3 μm or from 1.0 μm to 2 μm. Some larger fibers may be present in the layers and act as a scaffold. Such scaffold fibers may be as large as 15 μm or up to 20 The portion of the stack that is intended to act as a loading layer may include a combination of features, which may be present in each fiber layer within the loading layer or be present in separate fiber layers within the loading layer. For example, the loading layer may include a layer or layers constructed based on sieving capability of and another layer or layers constructed based on affinity or sieving of a different size or type of contaminant.

According to an embodiment, the layers of the composite media are under various levels of compression. That is, the composite media includes a first plurality of fiber layers that are under a first level of compression and a second plurality of fiber layers that are under a second level of compression different from the first level of compression. The composite media may further include one or more pluralities of fiber layers with a third, fourth, and subsequent levels of compression. The layers may be compressed together during the manufacturing of the stack or by using a housing (e.g., filter housing) that compresses stacks with various levels of compression. Referring now to FIG. 4, schematic cross-sectional view of a filter 1′ is shown with a housing 4 that applies various levels of compression to stacks 61, 62, 63 of layers. The number of stacks and the layers shown in each stack 61, 62, 63 is for illustrative purposes only and is not representative of the actual number of layers. Each stack 61, 62, 63 may include 20 or more layers. The first stack 61 may be under a first level of compression, the second stack 62 may be under a second level of compression, and third stack 63 may be under a third level of compression. The levels of compression may successively increase from the inlet end 41 to the outlet end 42 of the filter 1′. In some embodiments, the composite media or the filter that includes multiple composite media includes a gradient of compression levels. A higher level of compression may result in a smaller effective pore size of the stack. By applying different levels of compression, a different effective pore size of the various stacks may be achieved. In some embodiments, a gradient of compression levels is used to achieve a gradient of effective pore size.

The following is a list of exemplary embodiments according to the present disclosure.

According to embodiment 1, a composite media comprises a stack comprising 20 or more layers, each layer comprising:

a substrate comprising a first major surface and an opposing second major surface; and

a fiber layer deposited onto the first major surface of the substrate and comprising polymeric fibers with a diameter of 100 nm to 1.5 μm,

each fiber layer independently having a thickness of 5 μm to 100 μm, and each layer independently having a P95 pore size of 0.1 μm to 10 μm.

Embodiment 2 is the composite media of embodiment 1, wherein the substrate comprises a non-woven substrate.

Embodiment 3 is the composite media of embodiment 1 or 2, wherein the substrate comprises a membrane.

Embodiment 4 is the composite media of any one of embodiments 1 to 3, wherein the stack comprises a layer comprising two or more fiber layers, optionally wherein each layer of the stack comprises two or more fiber layers.

Embodiment 5 is the composite media of embodiment 4, wherein the stack comprises a layer comprising a second fiber layer deposited on the second major surface of the substrate.

Embodiment 6 is the composite media of any one of embodiments 1 to 5, wherein each layer independently has a P95 pore size of 0.1 μm or greater, 0.3 μm or greater, 0.5 μm or greater, 0.7 μm or greater, or 1.0 μm or greater. Each layer may independently have a P95 pore size of 10 μm or less, 7.5 μm or less, 5.0 μm or less, 3.0 μm or less, 2.0 μm or less, or 1.5 μm or less. The P95 pore size of the layer may range from 0.1 μm to 10 μm, 0.5 μm to 5.0 μm, from 0.7 μm to 2.0 μm, from 0.8 μm to 1.5 μm, or from 1.0 μm to 1.5 μm.

Embodiment 7 is the composite media of any one of embodiments 1 to 6, wherein the pore sizes of the fiber layers form a gradient of pore sizes throughout the stack.

Embodiment 8 is the composite media of embodiment 7, wherein the composite media has an inlet side and an outlet side, and the gradient of pore sizes extends from a largest pore size of 50 μm adjacent the inlet side to a smallest pore size of 0.1 μm adjacent the outlet side.

Embodiment 9 is the composite media of any one of embodiments 1 to 8, wherein the stack has a P95 pore size of 0.01 μm or greater, 0.05 μm or greater, 0.1 μm or greater, 0.3 μm or greater, 0.5 μm or greater, 0.75 μm or greater, or 1.0 μm or greater. The stack may have a P95 pore size of 2.5 μm or less, 2.2 μm or less, 2.0 μm or less, 1.75 μm or less, or 1.5 μm or less, of 1.0 μm or less. The P95 pore size of the stack may be from 0.01 μm to 2.5 μm, from 0.1 μm to 2.0 μm, or from 0.5 μm to 1.5 μm.

Embodiment 10 is the composite media of any one of embodiments 1 to 9, wherein the stack comprises 50 or more, 100 or more, 200 or more, 300 or more, or 500 or more layers. The stack may include 3000 or fewer, 2500 or fewer, 2000 or fewer, 1500 or fewer, or 1000 or fewer layers. The number of layers in the stack may be 100 to 3000 layers, 300 to 2000 layers, or 500 to 1000 layers.

Embodiment 11 is the composite media of any one of embodiments 1 to 10, wherein the stack comprises a first fiber layer with a first composition and a second fiber layer with a second composition different from the first composition.

Embodiment 12 is the composite media of embodiment 11, wherein fiber layers of the stack form a gradient of chemical composition throughout the stack.

Embodiment 13 is the composite media of any one of embodiments 1 to 10, wherein each of the layers in the stack has the same composition and structure.

Embodiment 14 is the composite media of any one of embodiments 1 to 13, wherein the stack comprises a first plurality of layers under a first level of compression and a second plurality of layers under a second level of compression different from the first level of compression.

Embodiment 15 is the composite media of embodiment 14, wherein pluralities of layers are under a gradient of compression levels throughout the stack.

Embodiment 16 is the composite media of any one of embodiments 1 to 15, wherein the composite media has an initial water flux of 100 mL/cm²/hour/kPa or greater, 120 mL/cm²/hour/kPa or greater, 150 mL/cm²/hour/kPa or greater, 175 mL/cm²/hour/kPa or greater, or 200 mL/cm²/hour/kPa or greater. The initial water flux may be 350 mL/cm²/hour/kPa or less, 300 mL/cm²/hour/kPa or less, or 250 mL/cm²/hour/kPa or less. The initial water flux may be from 100 mL/cm²/hour/kPa to 350 mL/cm²/hour/kPa or from 120 mL/cm²/hour/kPa to 300 mL/cm²/hour/kPa.

Embodiment 17 is the composite media of any one of embodiments 1 to 16, wherein the fiber layer has a surface area to volume ratio of 1 μm⁻¹ or greater, 2 μm⁻¹ or greater, or 5 μm⁻¹ or greater. The fiber layer may have SAVR of 20 μm⁻¹ or less or 15 μm⁻¹ or less. The SAVR of the fiber layer may range from 1 μm⁻¹ to 20 μm⁻¹ or from 2 μm⁻¹ to 15 μm⁻¹.

Embodiment 18 is the composite media of any one of embodiments 1 to 17, wherein the polymeric fibers have a diameter of 100 nm or greater, 200 nm or greater, 300 nm or greater, 400 nm or greater, or 500 nm or greater. The polymeric fibers may have a diameter of 1.5 μm or less, 1.2 μm or less, or 1.0 μm or less. The polymeric fibers may have a diameter ranging from 100 nm to 1.5 μm, from 300 nm to 1.2 μm, or from 500 nm to 1.0 μm.

Embodiment 19 is the composite media of any one of embodiments 1 to 18, wherein each fiber layer of the stack may independently have a thickness of 5 μm or greater, 10 μm or greater, 25 μm or greater, or 50 μm or greater. Each fiber layer may independently have a thickness of 100 μm or less, 80 μm or less, or 60 μm or less. The thickness of the fiber layer may range from 5 μm to 100 μm, 10 μm to 80 μm, or from 20 μm to 60 μm. The thickness of each of the fiber layers is not necessarily the same throughout the stack. Different fiber layers within the stack may have different thicknesses. The fiber layers within the stack may form a gradient of fiber layer thicknesses extending throughout the stack.

Embodiment 20 is the composite media of any one of embodiments 1 to 19, wherein the substrate has a thickness of 50 μm or greater, 100 μm or greater, or 500 μm or greater. The substrate may have a thickness of 1000 μm or less, 750 μm or less, or 500 μm or less. The thickness of the substrate may range from 50 μm to 1000 μm, or from 100 μm to 500 μm. The total thickness of the layer (combined substrate and fiber layer) may be from 55 μm to 1100 μm or from 150 μm to 600 μm.

Embodiment 21 is the composite media of any one of embodiments 1 to 20, wherein the composite media is constructed for filtering a liquid. The liquid may be an aqueous liquid. The liquid may be a non-aqueous liquid.

Embodiment 22 is a filter comprising a housing; and the composite media of any one of embodiments 1 to 21 disposed within the housing.

Embodiment 23 is the filter of embodiment 22, wherein the filter is a syringe filter.

Embodiment 24 is the filter of embodiment 22 or 23, wherein the filter housing is constructed to apply a first level of compression on a first plurality of layers and a second level of compression on a second plurality of layers, wherein the second level of compression is different from the first level of compression.

Embodiment 25 is the filter of embodiment 24, wherein pluralities of layers are under a gradient of compression levels throughout the stack.

Embodiment 26 is a composite media comprising a stack layers, each layer comprising a fiber layer deposited onto a surface of a substrate and comprising polymeric fibers with a diameter of 100 nm to 1.5 μm, each fiber layer independently having a thickness of 5 μm to 100 μm, and each layer independently having a P95 pore size of 0.1 μm to 10 μm, wherein at least two of the layers comprise a first type of fiber layer adjacent a second type of fiber layer, and wherein the first type of fiber layer of a first layer is not fully aligned with the first type of fiber layer of a second layer.

Embodiment 27 is the composite media of embodiment 26, wherein the first and second types of fiber layers differ by their solidity, fiber size, pore size, chemical composition, or a combination thereof.

Embodiment 28 is the composite media of embodiment 26 or 27, further comprising one or more of the features of embodiments 1 to 21.

Embodiment 29 is the composite media of any one of embodiments 26 to 28, disposed in a filter housing, optionally wherein the filter is a syringe filter.

Embodiment 30 is the composite media of embodiment 29, wherein the filter housing is constructed to apply a first level of compression on a first plurality of layers and a second level of compression on a second plurality of layers, wherein the second level of compression is different from the first level of compression, optionally wherein pluralities of layers are under a gradient of compression levels throughout the stack.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A composite media comprising:
a stack comprising 20 or more layers, each layer comprising:
a substrate comprising a first major surface and an opposing second major surface; and
a fiber layer deposited onto the first major surface of the substrate and comprising polymeric fibers with a diameter of 100 nm to 1.5 μm,
each fiber layer independently having a thickness of 5 μm to 100 μm, and each layer independently having a P95 pore size of 0.1 μm to 10 μm.

2. The composite media of claim 1, wherein the substrate comprises a non-woven substrate.

3. The composite media of claim 1, wherein the substrate comprises a membrane.

4. The composite media of claim 1, wherein the stack comprises a layer comprising two or more fiber layers.

5. The composite media of claim 4, wherein the stack comprises a layer comprising a second fiber layer deposited on the second major surface of the substrate.

6. The composite media of claim 1, wherein the pore size of the layers is from 0.5 μm to 5.0 μm.

7. The composite media of claim 1, wherein the pore sizes of the fiber layers form a gradient of pore sizes throughout the stack.

8. The composite media of claim 7, wherein the composite media has an inlet side and an outlet side, and the gradient of pore sizes extends from a largest pore size of 50 μm adjacent the inlet side to a smallest pore size of 0.1 μm adjacent the outlet side.

9. The composite media of claim 1, wherein the stack has a pore size of 0.01 μm to 2.5 μm.

10. The composite media of claim 1, wherein the stack comprises 100 to 3000 layers.

11. The composite media of claim 1, wherein the stack comprises a first fiber layer with a first composition and a second fiber layer with a second composition different from the first composition.

12. The composite media of claim 11, wherein fiber layers of the stack form a gradient of chemical composition throughout the stack.

13. The composite media of claim 1, wherein each of the layers in the stack has the same composition and structure.

14. The composite media of claim 1, wherein the stack comprises a first plurality of layers under a first level of compression and a second plurality of layers under a second level of compression different from the first level of compression.

15. The composite media of claim 14, wherein pluralities of layers are under a gradient of compression levels throughout the stack.

16. The composite media of claim 1, wherein the composite media has an initial water flux of 150 mL/cm²/hour/kPa to 300 mL/cm²/hour/kPa.

17. The composite media of claim 1, wherein the fiber layer has a surface area to volume ratio of 1 μm⁻¹ or greater.

18. A filter comprising:
a housing; and
the composite media of claim 1 disposed within the housing.

19. The filter of claim 18, wherein the filter is a syringe filter.

20. The filter of claim 18, wherein the filter housing is constructed to apply a first level of compression on a first plurality of layers and a second level of compression on a second plurality of layers, wherein the second level of compression is different from the first level of compression.

21. The filter of claim 20, wherein pluralities of layers are under a gradient of compression levels throughout the stack.
